(19) United States
(12) Patent Application Publication
(10) Pub. No.: US 2018/0172707 A1
(43) Pub. Date: Jun. 21, 2018

(54) DIAGNOSTIC TARGET

(71) Applicant: THE UNIVERSITY OF LINCOLN, Lincoln (GB)

(72) Inventors: Michael CHRISTIE, Lincolnshire (GB); Kerry MCLAUGHLIN, Berkshire (GB); Carolyn JOHNSON, Winchester, Hampshire (GB); Aarthi RAVISHANKAR, Greater Manchester (GB)

(21) Appl. No.: 15/579,042

(22) PCT Filed: Jun. 1, 2016
(86) PCT No.: PCT/GB2016/051597

§ 371 Date: Dec. 1, 2017

(30) Foreign Application Priority Data
Jun. 3, 2015 (GB) 1509575.5

(51) Int. Cl. G01N 33/68; A61P 3/10; A61K 38/17; C07K 14/47

(52) U.S. Cl. CPC G01N 33/6893; A61P 3/10; G01N 2800/042; C07K 14/47; A61K 38/1709

(57) ABSTRACT

A method for the diagnosis of Type 1 diabetes, or a predisposition towards Type 1 diabetes, or to monitor the efficacy of a therapy to prevent or treat Type 1 diabetes, said method comprising contacting a sample from a subject with a reagent selected from Tetraspanin-7 or a fragment, or a modified form thereof, and detecting an interaction indicative of the presence of an autoimmune response to Tetraspanin-7. Tetraspanin-7 is now understood to be the protein recognised by Glima 38 specific antibodies. Reagents and kits for use in the method and therapies associated with these form further aspects of the invention.

The present invention relates to a method for diagnosing Type 1 diabetes, or a predisposition to the development of Type 1 diabetes in subjects, as well as to kits and reagents for use in the method. Therapies based upon said reagents are also described and claimed.

### BACKGROUND TO THE INVENTION

The defining feature of Type 1 diabetes is the presence of autoimmunity to components of pancreatic islets that results in a destructive inflammation within the islets of affected patients. The inflammation results in a specific loss of insulin-secreting pancreatic beta cells occurring over a period of years, culminating in an inability to control blood sugar levels and a clinical diagnosis of diabetes. Disease occurs primarily in individuals expressing genes linked to disease, primarily within the class II region of the major histocompatibility complex (including HLA-DR3, HLA-DR4, HLA-DQ8), although expression of these alleles per se does not confer high risk for disease. Autoimmunity in the disease can be detected by assay for the presence of circulating autoantibodies to pancreatic islet component, initially performed by indirect immunofluorescence on frozen sections of human pancreas. As the molecular targets of autoimmune responses in Type 1 diabetes have been identified, sensitive, specific and high throughput assays to detect the presence of autoantibodies to individual target autoantigens have been developed that greatly facilitates the assessment of autoimmunity in individuals, both to assist in a clinical diagnosis of Type 1 diabetes and to identify individuals at risk for disease for entry into disease prevention trials. There is now good evidence from both animal studies and human trials that Type 1 diabetes can be prevented in individuals identified as at risk. Hence a range of therapies to interfere with immune responses in animal models of Type 1 diabetes such as the NOD mouse have proved effective in preventing disease development in the animals and administration of general immunosuppressive agents targeted at T-cells (cyclosporine, anti-CD3 antibodies) or B-cells (anti-CD20 antibodies) to recent onset Type 1 diabetic patients slows the continued loss of pancreatic beta cell function that occurs in the months following disease diagnosis. Unfortunately general immunosuppression leaves the individual at risk of immunodeficiency and is unlikely to represent a universal approach to diabetes prevention. Instead, there is a focus of current research on the development of procedures to interfere specifically in the immune responses that cause disease. This requires knowledge of the major targets of the immune response in human Type 1 diabetes, both for assessment of diabetes risk to identify individuals for whom immunotherapy is appropriate, and for the design of procedures to block immune responses to specific targets of the autoimmune response in the disease.

It is now evident that there is no single autoimmune target common to all Type 1 diabetic patients and that individuals differ in the antigen specificity of autoimmune responses that develop in their disease. Four major autoantigens have been identified in Type 1 diabetes on the basis of presence of autoantibodies at and before onset of disease: insulin, glutamate decarboxylase, IA-2 and ZnT8.

Autoantibodies to a fifth major autoantigen, a hydrophobic membrane glycoprotein named Glima 38, have been detected in a substantial proportion of Type 1 diabetic patients and the utility of this as a diagnostic marker has been described (EP0693183). However, the molecular identity of Glima 38 has for many years remained elusive, which has hampered studies to characterise autoimmunity to the protein in the disease and to develop sensitive and specific assays for autoantibody detection.

There appear to be many reasons for the difficulties associated with the isolation and purification of this particular protein. For instance, the protein is found only at very low abundance in pancreatic islets, and substantial quantities of pancreatic islet material for purification of islet autoantigens are difficult to obtain.

Furthermore, the protein is very hydrophobic and therefore difficult to solublise and purify, since hydrophobic peptides are difficult to elute from gels for identification by techniques such as LC-MS/MS.

In addition, the only antibodies available for immunoaffinity purification of the protein are autoantibodies in Type 1 diabetic patients' sera which are present at very low concentrations and heavily contaminated with other antibody specificities. There has been no reliable method to monitor the activity of the protein in different fractions generated during the purification process and methods for protein sequencing have, until recently, lacked the sensitivity required for identification of proteins at the concentrations recovered after immunoaffinity purification with patients' autoantibodies.

At present, any assay which seeks to determine the presence of Glima 38 antibodies involves complex procedures such as immunoprecipitation of radiolabeled islet cell proteins.

Autoimmunity to all major autoantigens (including Glima 38) first appears within the first 5 years of life in at risk individuals, with individual immune responses developing sequentially rather than simultaneously. Autoimmunity in the disease is therefore progressive, with the order of appearance of autoimmune responses to individual antigens differing between individuals, and diversification of the immune response being essential for disease progression; individuals who develop autoimmunity to only single autoantigens rarely develop disease. The optimum strategy for assessing disease risk is the screening of individuals for the presence of autoantibodies to multiple islet autoantigens.

The inclusion of Glima 38 in the ‘panel’ would therefore be expected to improve the accuracy and sensitivity of disease prediction, provide a full description of the major autoimmune responses that are developing in that individual and would guide the selection of autoantigen-specific immunotherapeutics to prevent the disease.

However, determination of the molecular identity of Glima 38 is critical for optimum Type 1 diabetes risk assessment and disease prevention.

The applicants have, using a complex strategy, positively identified the protein that forms the basis of Glima 38 and is recognised by anti-Glima 38 antibodies, as a result, a range of new diagnostic tests and therapies are possible.

### SUMMARY OF THE INVENTION

According to the present invention there is provided a method for the diagnosis of Type 1 diabetes, or a predisposition towards Type 1 diabetes, or to monitor the efficacy of a therapy to prevent or treat Type 1 diabetes, said method comprising contacting a sample from a subject with a reagent selected from Tetraspanin-7 or a fragment, or a modified form thereof, and detecting an interaction indicative of the presence of an autoimmune response to Tetraspanin-7.

Tetraspanin-7 is a hydrophobic 4-transmembrane domain membrane glycoprotein expressed in neuroendocrine tissues. It comprises a 27 kDa protein with 5 potential glycosylation sites. The theoretical pI of Tetraspanin-7 is 7.24.

```
It is highly conserved amongst species. The human sequence of
SEQ ID NO 1
                10 20 30 40 50
MASRRMETKP VITCLKTLLI IYSFVFWITG VILLAVGVWG KLTLGTYISL
                60 70 80 90 100
IAENSTNAPY VLIGTGTTIV VFGLFGCFAT CRGSPWMLKL YAMFLSLVFL
              110 120 130 140 150
AELVAGISGF VFRHEIKDTF LRTYTDAMQT YNGNDERSRA VDHVQRSLSC
              160 170 180 190 200
CGVQNYTNWS TSPYFLEHGI PPSCCMNETD CNPQDLHNLT VAATKVNQKG
              210 220 230 240
CYDLVTSFME TNMGIIAGVA FGIAFSQLIG MLLACCLSRF ITANQYEMV
The mouse sequence is highly homologous thereto, and is of SEQ
ID NO 2:
                10 20 30 40 50
MASRRMETKP VITCLKTLLI IYSFVFWITG VILLAVGVWG KLTLGTYISL
                60 70 80 90 100
IAENSTNAPY VLIGTGTTIV VFGLFGCFAT CRGSPWMLKL YAMFLSLVFL
              110 120 130 140 150
AELVAGISGF VFRHEIKDTF LRTYTDAMQN YNGNDERSRA VDHVQRSLSC
              160 170 180 190 200
CGVQNYTNWS SSPYFLDHGI PPSCCMNETD CNPLDLHNLT VAATKVNQKG
              210 220 230 240
CYDLVTSFME TNMGIIAGVA FGIAFSQLIG MLLACCLSRF ITANQYEMV
```

The applicants have determined that Tetraspanin-7 is the basic form of Glima 38 and is recognised by Glima 38 specific antibodies. As a result, effective diagnostic tests for damaging or potentially damaging autoimmune responses that are or may give rise to diabetes can be produced.

In reagent used in the method of the invention, such as Tetraspanin-7, in particular of SEQ ID NO 1 may be prepared synthetically, for example using recombinant technology for use in the diagnostic tests of the invention. Tetraspanin-7 used in the method of the invention will typically be in isolated or pure form in which it is free of some and preferably all of the other components found in Glima 38. It may be glycosylated or it may be unglycosylated. However, the reagent used is one which binds to Glima 38 antibodies.

As used herein, the expression ‘fragment’ refers to any portion of the given amino acid sequence, which has a similar overall activity. In particular, fragments will comprise one or more epitopes (epitopic fragments) and so be recognisable by antibodies for Tetraspanin-7. Fragments may comprise more than one portion from within the full length protein, joined together. Portions will suitably comprise at least 5 and preferably at least 10 consecutive amino acids from the basic sequence.

Suitable fragments will be deletion mutants suitably comprising at least 20 amino acids, and more preferably at least 100 amino acids in length. They include small regions from the protein or combinations of these.

In particular, any fragments will lack one or more hydrophobic regions, such as one or more of the transmembrane domains of Tetraspanin-7, in order to improve the solubility of the protein during the assay. The transmembrane domains include amino acids 17-40, 57-75, 87-112 and 214-234.

Also as used herein the expression “modified form” refers to peptides or proteins which are homologous to the basic Tetraspanin-7 protein or fragment, but which differ from the base sequence from which they are derived for example by addition of extra sequences or elements, or that one or more amino acids within the sequence are substituted for other amino acids. Again, the ‘modified form’ will have similar activity and in particular similar immunogenic properties to the basic protein or fragment. Additional sequences may be provided in order to provide further properties, for example to facilitate purification of recombinant proteins, including tag sequences such as a GST, HIS or FLAG tags, or to allow immobilisation of the reagent onto a support or other surface for the purposes of detection. Additional sequences or elements that may be used to enhance detection will vary depending upon the particular detection technique involved. For example, they may include label sequences, for example enzyme labels such as horseradish peroxidase or alkaline phosphatase that give rise to detectable signals as a result of enzymatic activity, luminescent labels such as luciferase or fluorescent labels such as fluorescein or derivatives thereof. Alternatively, where detection is carried out using ³⁵S methionine labelling technique, an additional sequence may be included, which has a high methionine content so that any signal is amplified.

Additional sequences or elements may be linked to the reagent covalently or may be conjugated or complexed thereto by other means.

Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the immunoreactivity of the polypeptide. Suitably variants will be at least 60% identical, preferably at least 70%, even more preferably 80% or 85% and, especially preferred are 90%, 95% or 98% or more identity.

Identity in this instance can be judged for example using the BLAST program or the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

The discovery that Tetraspanin-7 is the basis of the Glima 38 protein is surprising. The fact that it has taken nearly 20 years to identify this protein is indicative of the level of difficulty associated with its purification. The applicants found no less than 31 different 38 kDa proteins within a preparation of brain membrane glycoproteins, immunoaffinity purified using Glima 38 antibodies from Type 1 diabetic patients. It has only been the development of a novel and complex strategy as outlined further below, that the problem has been resolved.

Suitable samples for use in the method of the invention include blood, plasma or serum samples.

In a particular embodiment, the particular autoimmune response detected in the method is indicated by the presence of autoantibodies to Tetraspanin-7 in the sample. Suitably, the level of these antibodies are measured and compared to a normal reference range, for example as found in a healthy individual. Methods for detecting specific antibodies in a sample such as blood, serum or plasma sample are well known in the art, and include assays such as radioimmunoprecipitation assays, ELISAs, time-resolved fluorescence assays and luminescence assays including for example luminescence immunoprecipitation assays.

The basis of these techniques is that the samples are contacted with the reagent such that any Tetraspanin-7 specific antibodies present in the sample will bind to the reagent. This binding interaction may then be detected. In a particular embodiment, the reagent comprises a modified form of Tetraspanin-7 or a fragment thereof, wherein the modification is designed to increase the efficiency of detection of antibody binding. Suitable modified forms are as described above.

In particular, the modified form is one in which the reagent is linked or complexed to a further protein or peptide which increases the efficiency of antibody detection. Suitable further proteins or peptides includes labels (such as radioactive labels, fluorescent labels, luminescent labels or enzymatic labels) as well as proteins or peptides with a high methionine content to facilitate detection in ³⁵S methionine labelling.

In a particular embodiment, the reagent is Tetraspanin-7 of SEQ ID NO 1. It may be free of glycosylation. However, in an alternative embodiment, the Tetraspanin-7 may comprise a level of glycosylation, in particular if this enhances recognition by Tetraspanin-7 specific antibodies.

In a particular embodiment, the Tetraspanin-7-specific antibodies are detected using a radioimmunoprecipitation assay. This is a well-known technique for detection of autoantibodies in Type 1 diabetes, in which radiolabeled reagent is used to detect autoantigen specific antibodies in a sample such as a serum sample. The reagent is allowed to react with the serum and then precipitated using a special reagent such as Protein A sepharose beads. The bound radiolabeled immunoprecipitate may then be analyzed by liquid scintillation or gamma counting. An example of such an assay is found in for example Hatfield E C, et al. Diabetologia 40(11):1327-1333.

A variation of this technique is a luminescence immunoprecipation, as illustrated in the luminescence immunoprecipitation assay described in Burbelo P D, et al. (2008) Diabetes Care 31(9):1824-1826. In this assay, the use of radiolabels is avoided since the reagent would comprise Tetraspanin-7 or a fragment thereof, fused to a luminescence producing protein such as luciferase.

In an alternative embodiment, an ELISA may be used. In this case, the reagent may be immobilised on a support such as a well in a plate or a membrane such as a nitrocellulose membrane as found in a lateral flow type assay. The sample is contacted with the sample such that any specific antibodies are bound to the reagent forming an immobilised antigen-antibody complex. Residual sample is then removed and immobilised complex is detected, for example using secondary antibodies which are specific for the antibodies of the target, such as human antibodies, and which may give rise to a detectable signal, such as a visible signal, either directly on or addition of further reagents such as horseradish peroxidase. Such methods avoid the use of radiolabels.

In an alternative embodiment of the method of the invention, the autoimmune response detected is indicated by the presence of Tetraspanin-7 specific T-cells. Again, assays for specific T-cells are known in the art, and may include T cell proliferation assays, a binding assay using soluble MHC tetramers, a binding assay using soluble T cell receptors, an ELISPOT assay or an assay based upon cytokine detection such as an intracellular cytokine staining assay or acytokine secretion assay.

In a particular embodiment, the assay is a MHC-tetramer binding assay. Soluble MHC molecules containing a biotinylated protein domain are mixed with the reagent as described above, in particular Tetraspanin-7 or a fragment thereof, forming reagent-MHC (pMHC) complexes. These complexes are then bound to a fluorescently tagged streptavidin complex with high affinity as a result of the presence of the biotin, forming a tetramer. The resulting pMHC-streptavidin-fluorophore tetramers are then added to the sample, such as a blood serum or plasma sample, whereupon they bind to any T-cells that are specific for both the MHC type and Tetraspanin-7. Once the tetramers are bound, T-cells may be stained with other fluorophores and the sample is washed to remove non-bound tetramers and ligands. The stained sample is then run through a flow cytometer for detection and sorting. The fluorophore on any bound tetramers provides a signal indicating the presence of Tetraspanin-7 specific T-cells. The strength of the signal obtained in this way provides an indication of the strength of the immune response.

Alternatively, Tetraspanin-7 specific T-cells are detected using an assay based upon cytokine detection. One example of such an assay uses intracellular cytokine staining (ICS) to detect the production and accumulation of cytokines within the endoplasmic reticulum after cell stimulation.

In this method, the reagent is used to activate T-cells in the sample. These are then treated with an inhibitor of protein transport (e.g. brefeldin A) to retain the cytokines within the cell. After washing, the cells are fixed in paraformaldehyde and permeabilized. After addition of a detectable anti-cytokine antibody, the cells can be analyzed by flow cytometry. A particular example of such a method is described by Foster B, et al. Curr Protoc Immunol Chapter 6: Unit 6 24.

In another assay, cytokine secretion is detected. In this assay, cytokines secreted by Tetraspanin-7 specific T-cells are captured and detected after stimulation with the reagent, using techniques such as flow cytometry, or after enrichment by a magnetic-based separation system or fluorescence-activated cell sorting. A particular cytokine secretion method is described for example in Campbell J D, et al. (2011) Clin Exp Immunol 163(1):1-10.

Suitably the method of the invention forms an element in a suite of tests aimed at detecting a range of diabetes markers. Thus in a particular embodiment, the method of the invention further comprises the steps detecting an immune response to one or more additional diabetes markers such as GAD, insulin, IA-2 or ZnT8. These further markers may be detected by any of the methods described above for the detection of the reagent of the invention.

Conveniently however, where a number of such markers are to be investigated, methods involving detection of specific antibodies may be preferred for ease of use.

The methods of the invention, may, in a particular embodiment, be used to detect the presence of Type 1 diabetes in a patient.

In another particular embodiment, they may be used to diagnose a predisposition to Type 1 diabetes, before clinical symptoms appear. In particular, if such tests can be carried out on samples from young patients, for example, those who may be genetically disposed towards diabetes, therapeutic interventions may be instigated that reduce the risk that disease develops further.

In yet another embodiment, the method of the invention is used to monitor the efficiency of a therapy to prevent or treat Type 1 diabetes. In this case, the method may be repeated over a period during which a subject is undergoing prophylactic or therapeutic treatment to determine whether the treatment is producing an effect on the autoimmune response of the patient, so that, if necessary, modification of the treatment may be made in accordance with normal clinical practice.

Kits for use in the method described above form a further aspect of the invention. In particular, such kits comprise a reagent selected from Tetraspanin-7 or a fragment, or a modified form thereof, and means for detecting an interaction indicative of the presence of an autoimmune response to Tetraspanin-7.

Suitable forms of the reagent are as described above. In particular the reagent is Tetraspanin-7, which may be glycosylated or unglycosylated. Alternatively, where the kit is intended for use in an MHC tetramer assay for Tetraspanin-7 specific T-cells, the reagent may comprise a modified form of Tetraspanin-7 for example, a complex such as a pMHC-streptavidin-fluorophore tetramers, where the ‘p’ represents the reagent.

The means for detecting an interaction indicative of the presence of an autoimmune response to Tetraspanin-7 provided in said kit will vary depending upon which assay is being carried out. Thus for example, in the case of an ELISA assay, the kit may further comprise secondary antibodies or immobilising agents that allow antigen-antibody interactions to be detected. but may also include buffers, detectable labels and reagents useful for reading the assay. These may also be useful in other types of assay and therefore included in the kit, together with soluble binding proteins (e.g. soluble MHC, soluble T cell receptors) amongst others.

Thus, Tetraspanin-7 and proteins and peptides based upon it may have a wide variety of applications in the diagnostic field. In yet a further aspect, the invention provides the use of Tetraspanin-7 or fragments, in particular epitopic fragments thereof or modified forms thereof for use in in-vitro methods of diagnosis or treatment of Type 1 diabetes or a predisposition towards Type 1 diabetes.

The identification of Tetraspanin-7 as a key factor in Type 1 diabetes development, may further lead to therapeutic options.

Thus in a further aspect, the invention provides Tetraspanin-7 or an epitopic fragment thereof or modified form thereof, for use in methods of prophylactic or therapeutic treatment of Type 1 diabetes or a predisposition towards Type 1 diabetes.

In particular, the invention may provide a method for preventing Type 1 diabetes, delaying the onset of Type 1 diabetes, or ameliorating autoimmunity in an individual with Type 1 diabetes (including ameliorating any one or more symptoms of the disease), comprising administering to an individual in need thereof, an agent that either (i) elicits a Tetraspanin-7-specific immune response that protects [beta] cells of the pancreatic islet in the patient; or (ii) targets Tetraspanin-7-specific T cells in the individual, and induces necrosis or apoptosis of the Tetraspanin-7-specific T cells; or (iii) induces tolerance of Tetraspanin-7 T cells in the individual; or (iv) depletes Tetraspanin-7 specific B-cells.

The means by which these therapies can be delivered will vary depending upon factors such as the nature of the patient, the disease state and the type of therapy envisaged. For example, in T-Cell or B-cell depletion therapy, the aim would be to destroy or inactivate malignant Tetraspanin-7 specific T or B cells in patients, while at the same time retaining protective T and B cell immunity. The reagents of the invention may therefore be coupled to reagents such as cytotoxins or antibody fragments and administered to the patient to target those cells for destruction. Alternatively, fragments or modified forms of Tetraspanin-7 may be administered as a form of therapeutic or prophylactic vaccine so as to prevent the onset of a damaging autoimmune response.

In some instances, the ‘agent’ used in these methods of treatment may comprise Tetraspanin-7 or fragments thereof, or modified forms of these.

Novel fragments and modified forms of Tetraspanin-7 useful in any of the above methods, form yet a further aspect of the invention.

Suitably, any agent used the above methods of treatment will be administered in the form of a pharmaceutical composition, in combination with a pharmaceutically acceptable carrier.

Thus in yet a further aspect, the invention provides a pharmaceutical composition comprising Tetraspanin-7 or fragments thereof, or modified forms of these in combination with a pharmaceutically acceptable carrier or excipient.

Suitable pharmaceutical compositions will be in either solid or liquid form. They may be adapted for administration by any convenient route, such as parenteral, oral or topical administration or for administration by inhalation or insufflation. The pharmaceutical acceptable carrier may include diluents or excipients which are physiologically tolerable and compatible with the active ingredient.

Parenteral compositions are prepared for injection, for example either subcutaneously or intravenously. They may be liquid solutions or suspensions, or they may be in the form of a solid that is suitable for solution in, or suspension in, liquid prior to injection. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

Oral formulations will be in the form of solids or liquids, and may be solutions, syrups, suspensions, tablets, pills, capsules, sustained-release formulations, or powders. Oral formulations include such normally employed excipients as, for example, pharmaceutical grades of mannitol, lactose, starch, magnesium stearate, sodium saccharin, cellulose, magnesium carbonate, and the like.

Topical formulations will generally take the form of suppositories or intranasal aerosols. For suppositories, traditional binders and excipients may include, for example, polyalkylene glycols or triglycerides; such suppositories may be formed from mixtures containing the active ingredient.

The amount of reagent administered will vary depending upon factors such as the precise nature of the inhibitor, the size and health of the patient, the nature of the condition being treated etc. in accordance with normal clinical practice.

Typically, a dosage in the range of from 1 μg-50 mg/Kg such as from 10 μg-1 mg/Kg would be expected to produce a suitable effect.

### DETAILED DESCRIPTION OF THE INVENTION

The invention will now be particularly described by way of example with reference to the accompanying drawings in which:

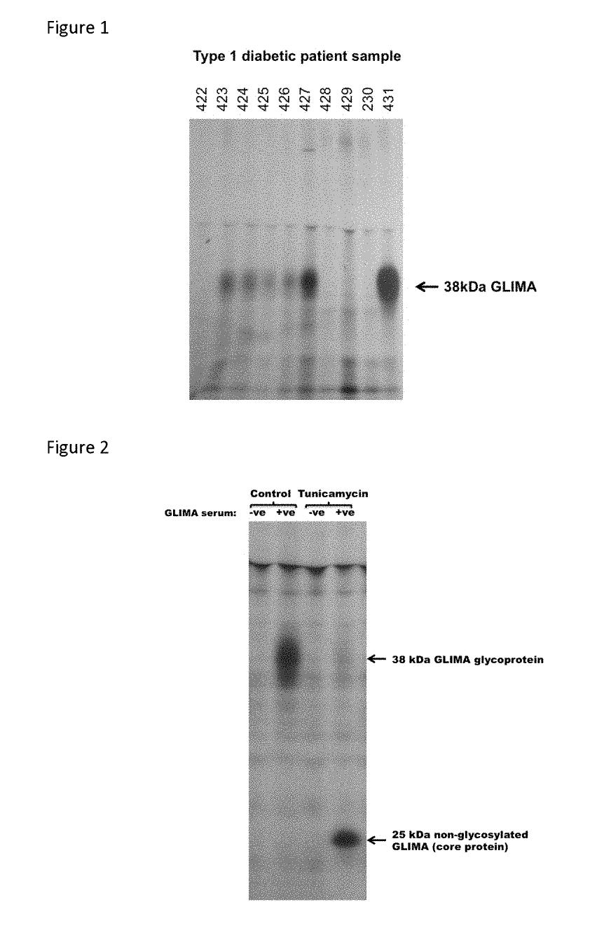

FIG. 1 shows an autoradiogram showing proteins immunoprecipated by antibodies in recent onset Type 1 diabetic patients. The location of the band representing the 38 kDa Glima 38 protein is marked by an arrow.

FIG. 2 is an autoradiograph showing the influence of tunicamycin on the mobility of Glima 38 on SDS-PAGE. RIN5AH cells were labelled with ³⁵S methionine in the absence or presence of tunicamycin before immunoprecipitation with sera negative or positive for Glima 38 antibodies, SDS-PAGE and autoradiography. The relative molecular masses of Glima 38 bands in the absence or presence of tunicamycin are marked.

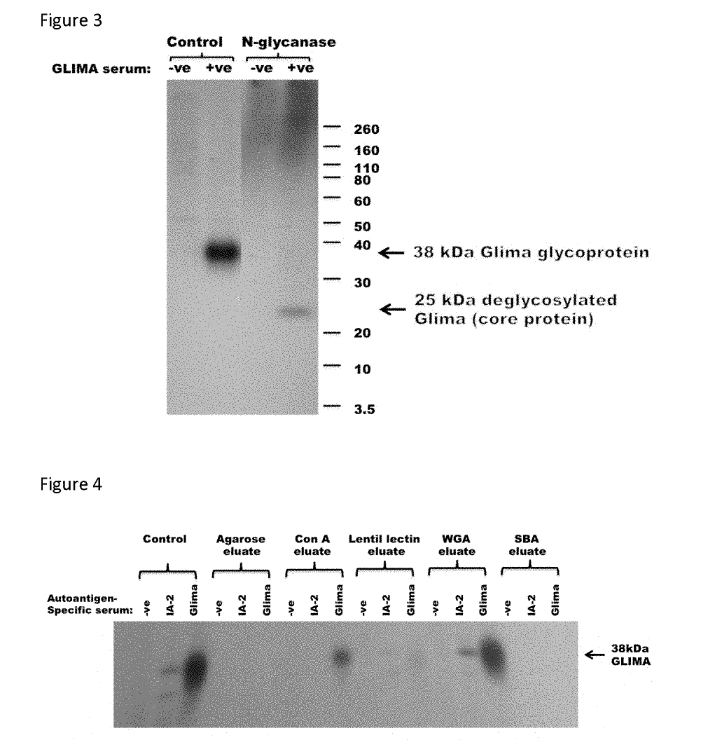

FIG. 3 is an autoradiograph showing the influence of N-glycanase on mobility of Glima 38 on SDS-PAGE. Extracts of ³⁵S methionine-labelled GT1.7 neuronal cells were incubated with sera negative or positive for Glima 38 antibodies and immune complexes captured on protein A Sepharose. Immunoprecipitated proteins were denatured by addition of SDS and incubated in the absence (control) or presence of N-glycanase for 18 hours before analysis by SDS-PAGE and autoradiography.

FIG. 4 is an autoradiograph showing binding of insulinoma cell IA-2 and Glima 38 to lectin-conjugated agarose. Radiolabelled extracts of RIN5AH insulinoma cells were prepared and incubated with unconjugated agarose or with agarose conjugated to different lectins as in the figure. Eluates from the agarose preparations were immunoprecipitated with normal control serum or Type 1 diabetic patients' sera positive for antibodies to IA-2/IA-2beta or Glima 38. Immune complexes were captured on protein A-Sepharose and subjected to SDS-PAGE and autoradiography. IA-2 antibody samples were treated with 0.1 mg/ml trypsin for 20 mins on ice prior to electrophoresis to generate 40 kDa and 37 kDa fragments of the autoantigens.

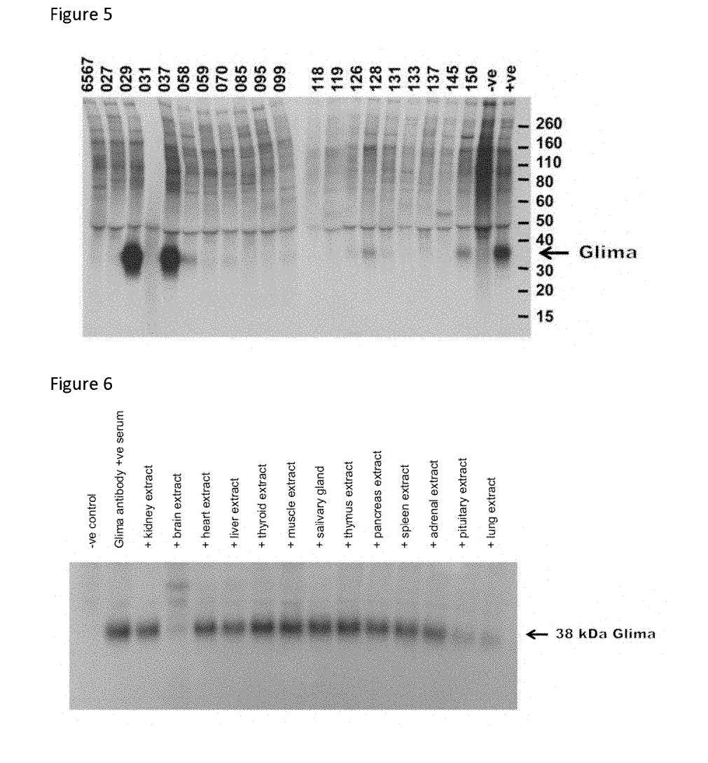

FIG. 5 is an autoradiogram of screen for Glima 38 antibody positive serum samples. The location of the Glima 38 band is marked.

FIG. 6 is an autoradiograph of tissue expression screen demonstrating competition for Glima 38 antibody binding by proteins in extracts of brain, pituitary and lung.

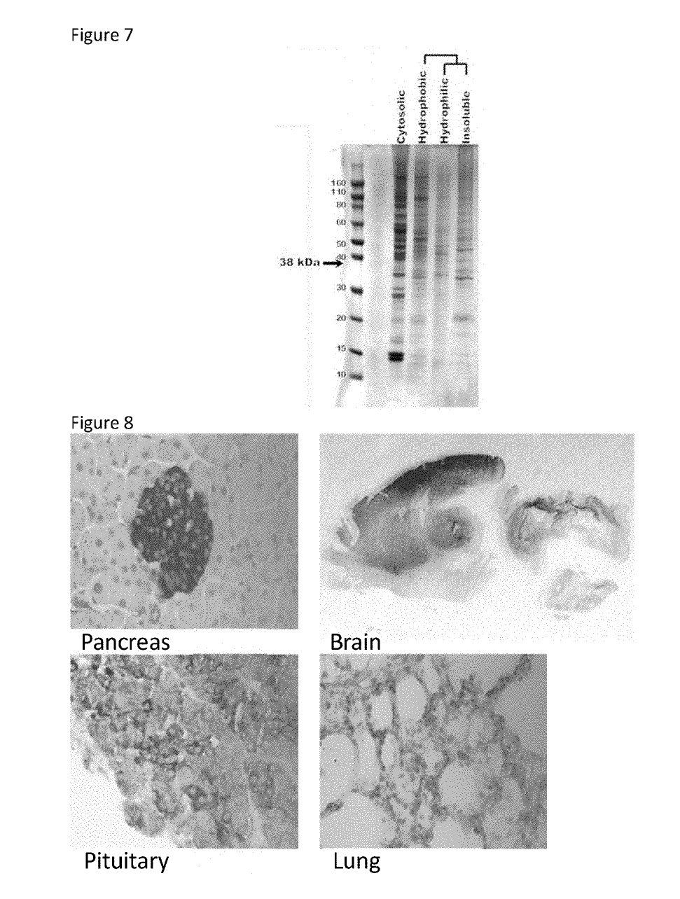

FIG. 7 shows a Coomassie-stained SDS polyacrylamide gel illustrating proteins present in cytoplasmic, hydrophobic detergent phase, hydrophilic detergent phase and detergent-insoluble fractions from mouse brain extracts.

FIG. 8 shows the results of immunohistochemical analysis of Tetraspanin-7 expression in rodent tissues.

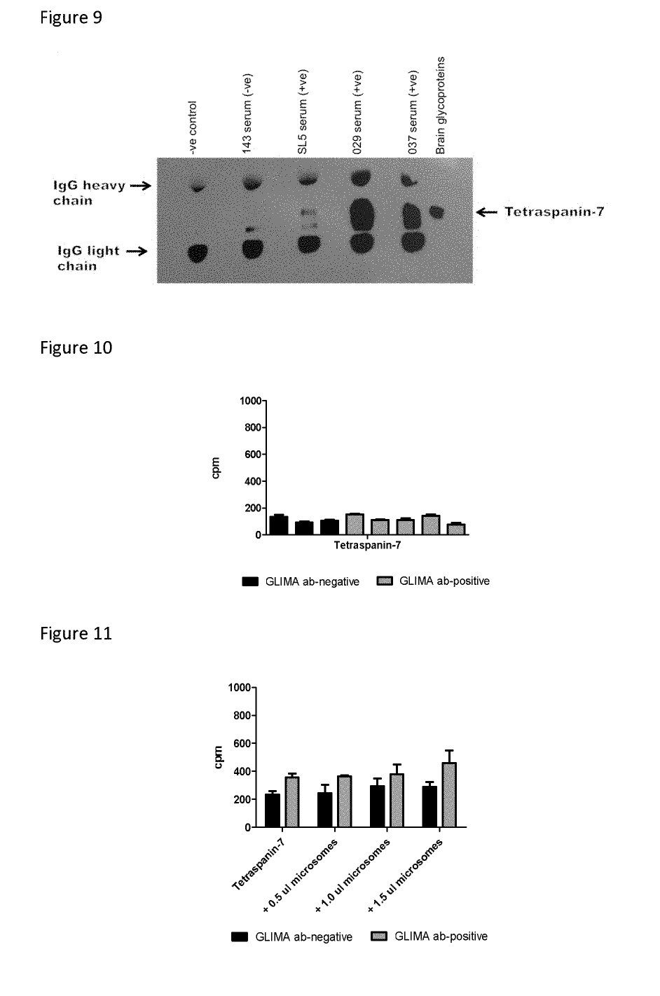

FIG. 9 shows Tetraspanin-7 labelling of western blots of mouse brain extracts immunoprecipitated by antibodies in sera from Glima 38 antibody negative and positive recent onset Type 1 diabetic patients. The location of Tetraspanin-7 in the brain glycoprotein preparation (last lane on western blot) is shown. The IgG heavy and light chains from the patients' serum samples were also detected on the blot as a consequence of cross-reactivity with the peroxidase-conjugated anti-rabbit antibody used for detection of primary antibody binding.

FIG. 10 is a graph showing immunoprecipitation of in vitro transcribed and translated Tetraspanin-7 by human patient sera evaluated by radioimmunoassay.

FIG. 11 is a graph showing immunoprecipitation of in vitro transcribed and translated Tetraspanin-7 by human patient sera evaluated by radioimmunoassay. Canine pancreatic microsomes were added at the indicated concentrations.

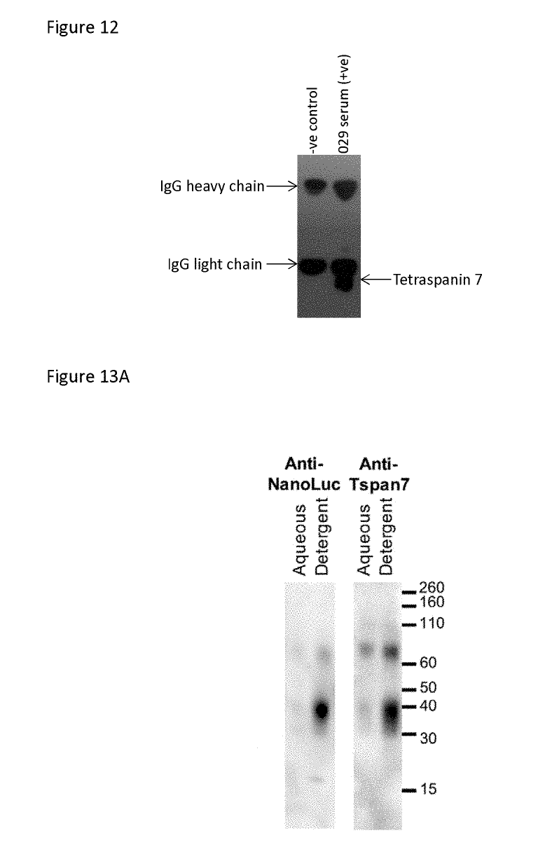

FIG. 12 shows Tetraspanin-7 labelling of western blots of Tetraspanin-7-transformed E. coli lysate immunoprecipitated by antibodies in sera from Glima 38 antibody negative and positive recent onset Type 1 diabetic patients. The IgG heavy and light chains from the patients' serum samples were also detected on the blot as a consequence of cross-reactivity with the peroxidase-conjugated anti-rabbit antibody used for detection of primary antibody binding. The recombinant Tetraspanin-7 migrates at approximately 20 kDa, lower than that of endogenous Tetraspanin-7, most likely due to a lack of post-translational modifications.

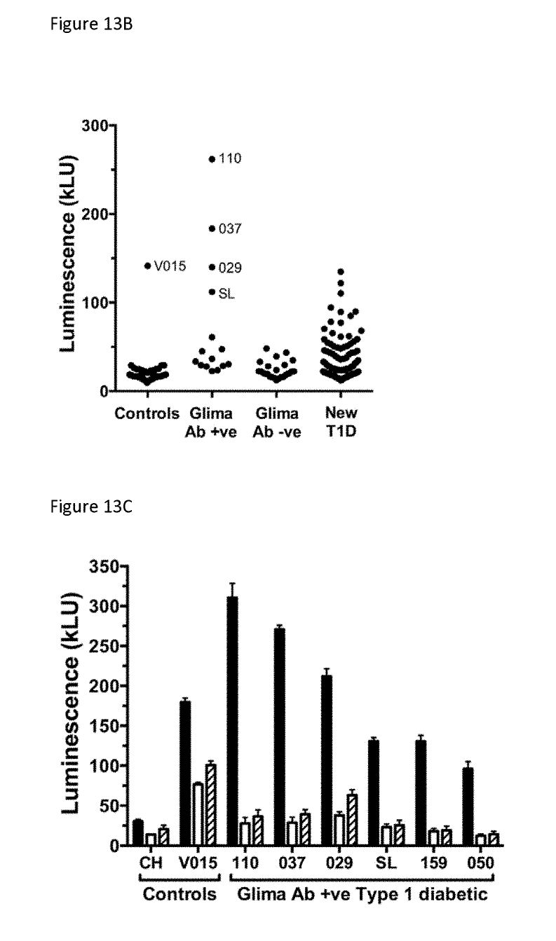

FIG. 13 shows the results of a luminescence-based immunoprecipitation system (LIPS) in which A: Tspan7 was expressed as a fusion protein with NanoLuc luciferase, and Triton X-114 extracts of cells were subject to heat-induced phase separation. Detergent and aqueous phases were subject to SDS-PAGE and Western blotting with antibodies to NanoLuc luciferase or Tspan7. The migration of molecular weight markers are shown (1023 3 Mr). B: Detergent extracts containing NanoLuc luciferase-tagged Tspan7 were immunoprecipitated with normal control sera (−ve) (n=30), sera from Glima antibody (Ab)-positive patients with type 1 diabetes (T1D) (n=15), and the sera of Glima antibody-negative patients with type 1 diabetes and luciferase activity associated with each immunoprecipitate determined by luminometry. Data are plotted as luciferase activity immunoprecipitated in kilo light units (kLU), and sample codes for control or diabetic individuals with high levels of antibodies are shown. C: Samples from control individuals or Glima antibody-positive patients with type 1 diabetes were tested for competitive binding by natural or recombinant Tetraspanin-7 in the LIPS by performing the immunoprecipitations in the absence (black bars) or presence of 150 mg of mouse brain extract (white bars) or 250 mg of lysates of E. coli expressing recombinant Tetraspanin-7 (hatched bars). Assays were performed in triplicate. The addition of brain and E. coli lysate significantly blocked antibody binding for all samples (P<0.0001; ANOVA with Dunnett correction for multiple comparisons), with the exception of control sample CH.

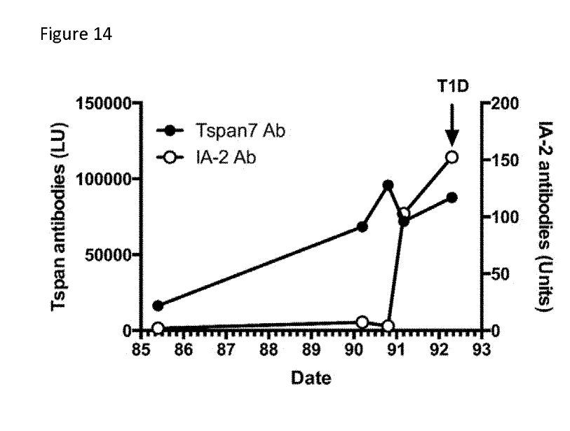

FIG. 14 is a graph showing the results of a LIPS assessment of the content of antibodies to Tetraspanin-7 (Tspan7 Ab) and the known diabetes marker, IA-2 (IA-2 AB), in historical sera samples taken between 1985 (‘85’) and 1992 (‘92’), from a patient who went on to develop diabetes (T1D) at the time of the last sample. Elevated levels of Tspan7 antibodies were detected more than 2 years before the development of diabetes and 1 year before the detection of IA-2 antibodies.

### EXAMPLE 1

Detection of Radiolabelled 38 kDa Protein (Glima 38) Expressed by Pancreatic Beta Cell or Hypothalamic Cell Lines Immunoprecipitated by Antibodies in Type 1 Diabetic Patients' Sera.

A 38 kDa islet membrane autoantigen in Type 1 diabetes (referred to as Glima-38) has been shown to be expressed in immortalised pancreatic beta cell and neuronal cell lines by immunoprecipitation from extracts of cells labelled with ³⁵S methionine (Aanstoot et al., (1996) J Clin Invest 97: 2772-2783 Roll U, et al. (2000) Diabetologia 43:598-608 1996). A similar approach was used to detect antibodies to Glima-38.

Pancreatic beta cell or hypothalamic cell lines RINm5F, betaTC or GT1.7 were cultured in Dulbecco's modified Eagle's medium (DMEM), containing 4,500 mg/l glucose and 10% fetal calf serum in 25 cm² or 75 cm² tissue culture flasks. Cells were passaged after removal from flasks by trypsinisation in 2.5 g/l trypsin, 0.2 g/l EDTA in Hank's Balanced Salt solution. Cells were plated in 25 cm² flasks for labelling endogenous proteins with ³⁵S methionine. The adherent cells were washed in labelling medium (methionine- and cysteine-free DMEM containing 2 g/l bovine serum albumin) and incubated in labelling medium (3 ml) containing 12 MBq ³⁵S-methionine for 7 hours at 37° C. The adherent cells were washed twice with 5 ml of complete DMEM containing 10% fetal calf serum and cells collected from flask with a cell scraper. Cells were washed with 10 mM Hepes, pH7.4, 150 mM NaCl, 10 mM benzamidine, flash frozen in liquid nitrogen and stored at −80° C.

The frozen cell pellet was extracted in 200 pI of extraction buffer containing 10 mM Hepes, pH 7.4, 150 mM NaCl, 10 mM benzamidine and 2% Triton X-114 for 2 hours on ice. After extraction, the cells were centrifuged at 15,000×g for 15 minutes at 4° C. The supernatant was collected and incubated at 30° C. for 3 minutes, followed by centrifugation at 3,000×g for 3 minutes to sediment a detergent phase containing amphiphilic membrane proteins. 10 mM Hepes, pH 7.4, 150 mM NaCl, 10 mM benzamidine (150 μl) was added to the detergent phase, the detergent pellet redissolved on ice and the phase separation repeated.

Radioactivity in a 1 μl aliquot of detergent phase fractions was quantified by liquid scintillation counting and fractions diluted to 1.25×10⁸ cpm/ml. The detergent phase fractions were pre-cleared of proteins binding non-specifically by incubation with 50 μl of normal human serum on ice for 2 h, followed by 45 minutes incubation with 50 μl of protein A-Sepharose at 4° C. Protein A-Sepharose was removed by centrifugation at 3,000×g and aliquots of supernatants incubated with 5 μl of test serum samples from Type 1 diabetic patients or non-diabetic healthy controls for 18 hours at 4° C.

Immune complexes were captured on 5 μl of protein A-Sepharose, incubating at 4° C. for 45 min, and Sepharose pellets washed three times with 1 ml of Immunoprecipitation Wash Buffer (10 mM Hepes, pH7.4, 150 mM NaCl, 10 mM benzamidine, 0.5 mM methionine, 100 mg/l bovine serum albumin, 0.5% Triton X-100) and once with water. Immunoprecipitates were eluted from protein A-Sepharose in 15 μl of sodium dodecyl sulphate-polyacrylamide gel electrophoresis (SDS-PAGE) sample buffer with heating at 90° C. for 5 min. Eluates were subjected to SDS-PAGE on 12% polyacrylamide gels and proteins fixed in gels in 40% v/v methanol, 2.5% v/v acetic acid. Gels were washed briefly in water and incubated in Enlightning autoradiographic enhancer (Perkin-Elmer) for 30 min. Gels were dried and contacted with X-ray film (Kodak BioMax MR film) for up to two weeks. After exposure, X-ray film was developed to detect radiolabelled proteins specifically immunoprecipitated by sera from Type 1 diabetic patients. The results are shown in FIG. 1. A band representing the 38 kDa Glima 38 protein was clearly visible.

Using this protocol, in a blinded analysis of autoantibodies in serum samples (Winnock F, et al. (2001) Diabetes Care 24: 1181-1186), autoantibodies were found in:
1. 38 of 100 (38%) of recent onset Type 1 diabetic patients selected from the Belgian Diabetes Registry;
2. 20 Of 39 (51%) of recent onset Type 1 diabetic patients aged <15 years of age at disease onset;
3. 8 of 23 (35%) non-diabetic siblings who progressed to Type 1 diabetes on follow up;
4. 0 of 100 (0%) non-diabetic healthy control subjects.

These results establish Glima 38 antibodies as important diagnostic and predictive markers of Type 1 diabetes that would be valuable for screening for at-risk subjects if the target antigen could be identified to allow development of commercial autoantibody assays.

### EXAMPLE 2

Glycosylation of Glima 38

To determine the glycosylation status of Glima 38, RIN5AH rat insulinoma cells were incubated in the presence of glycosylation inhibitors during metabolic labelling of endogenous proteins before extraction and immunoprecipitation of Glima as described in Example 1 above. RIN5AH cells were plated in 24-well plates to confluence and incubated in 1 ml labelling medium alone or in the presence of the N-glycosylation inhibitor tunicamycin (10 μg/ml) for 30 minutes at 37° C. before addition of 9 MBq ³⁵S methionine. Cells were labelled for 5 hours at 37° C. before harvesting, extraction and immunoprecipitation as in Example 1 above. Blocking of N-glycosylation with tunicamycin was found to reduce the relative molecular mass of the immunoprecipitated labelled autoantigen from 38,000 to approximately 25,000 (FIG. 2), indicating that the core polypeptide chain of Glima 38 is approximately 25 kDa.

To further evaluate glycosylation of Glima 38, RIN5AH insulinoma cells or GT1.7 neuronal cells were labelled with ³⁵S methionine and immunoprecipitated with serum samples from Type 1 diabetic patients determined to be positive for antibodies to Glima 38, or from negative control sera, and immune complexes captured on 5 μl of protein A Sepharose as described in Example 1 above. A 1 μl aliquot of 1% w/v SDS solution was added to the protein A Sepharose pellets and the samples heated to 95° C. for 3 minutes to denature immunoprecipitated glycoproteins. Sodium phosphate buffer (20 μl of 50 mM sodium phosphate pH 7.2 containing 0.5% Triton X-100) was added together with 400 mU of N-glycanase (Roche Diagnostics GmbH, Mannheim, Germany) and samples incubated for 18 hours at 37° C. to remove N-linked carbohydrate from immunoprecipitated glycoproteins. Enzyme reactions were stopped by addition of 20 μl of SDS-PAGE sample buffer and samples heated to 95° C. for 3 mins. Samples were centrifuged to remove protein A Sepharose and supernatants subjected to SDS-PAGE and autoradiography as described in Example 1 above. Consistent with the tunicamycin experiments, N-glycanase reduced the relative molecular mass of the immunoprecipitated Glima 38 to that of the core 25 kDa polypeptide chain of Glima 38 (FIG. 3).

The results of the experiments with glycosylation inhibitors and N-glycanase are consistent with Glima 38 being an N-glycosylated protein.

### EXAMPLE 3

Purification of Glima 38 Basic Protein

The applicants appreciated that glycosylation is a property that can be exploited to facilitate purification by lectin affinity chromatography for the purpose of protein identification. To determine which lectins are most appropriate for use in Glima 38 purification, Triton X-114 detergent phase fractions of 35S-methionine-labelled RIN5AH cells were prepared as described in Example 1 above. Detergent phase-partitioned proteins equivalent to 1.2×10⁷ cpm per sample were incubated with 50 μl of concanavalin A agarose (selective for glycoproteins with branched α-mannosidic structures), lentil lectin agarose (selective for glycoproteins with a fucosylated core region of bi- and triantennary complex type N-Glycans), wheat germ agglutinin agarose (selective for glycoproteins with N-acetyl glucosamine or sialic acid-rich carbohydrate) or soy bean agglutinin agarose (selective for glycoproteins with terminal N-acetyl galactosamine or galactose sugars) for 30 minutes at 4° C. with mixing. Beaded lectins with bound glycoproteins were washed 3 times with immunoprecipitation wash buffer and glycoproteins eluted in 2×50 μl of: 0.5M alpha methyl mannoside (concanavalin A- or lentil lectin-bound proteins), 0.5M N-acetyl glucosamine (wheat germ agglutinin-bound proteins) or 0.5M N-acetyl galactosamine (soy bean agglutinin-bound proteins. 20 μl of eluates were incubated with negative control sera, with IA-2 antibody-positive Type 1 diabetic patients' sera, or with patients' sera positive for antibodies to Glima 38 for 18 hours at 4° C.

Protein A Sepharose (5 μl) was added to all samples and subjected to SDS-PAGE and autoradiography as described in Example 1 above. The results demonstrated high recovery of both IA-2 and Glima 38 from wheat germ agglutinin agarose, lower recovery from concanavalin A agarose and negligible binding to lentil lectin or soy bean agglutinin (FIG. 4). Wheat germ agglutinin affinity chromatography is therefore valuable for the purpose of Glima 38 purification.

Patient samples were then screened to identify those high in autoantibodies suitable for affinity purification of Glima 38. For this purpose, the methodology of Example 1 was adapted to include a wheat germ agglutinin-agarose purification step. GT1.7 cells were labelled and extracted in Triton X-114 as described in Example 1 above and incubated on 25 μl wheat germ agglutinin-agarose on ice with frequent mixing for 30 minutes. The lectin agarose with bound glycoproteins was washed 3 times with 1 ml of immunoprecipitation wash buffer and glycoproteins eluted in 3×100 μl of 0.5M N-acetyl glucosamine in immunoprecipitation wash buffer. Radioactivity in 1 μl of eluate was quantified by liquid scintillation counting and diluted to 16×10⁶ cpm per ml. Aliquots of eluate (20 μl) were incubated with 5 μl of test sera for 18 hours at 4° C. and immune complexes captured on protein A Sepharose for SDS-PAGE and autoradiography as described in Example 1 above. Sera from 20 recent onset Type 1 diabetic patients were used in the antibody screen.

The results are shown in FIG. 5. Two patients (029 and 037) were identified as strongly positive for Glima 38 antibodies and used in subsequent experiments for Glima 38 immunoaffinity purification (see Example 5D below).

### EXAMPLE 4

Tissue Expression Screen

To obtain information on the tissue specificity of Glima 38 expression, competitive binding studies were performed using detergent extracts of normal mouse tissues as unlabelled competitors with 35S-methionine-labelled Glima 38 from GT1.7 cells for binding to Glima 38 antibodies in serum from Type 1 diabetic patient 029. Normal mouse tissues (kidney, brain, heart, liver, thyroid, muscle, salivary gland, thymus, pancreas, spleen, adrenal, pituitary and lung) were dissected and flash frozen in liquid nitrogen for storage before extraction. Tissues were thawed and homogenised in 10 mM Hepes, pH 7.4, 0.25 M sucrose, 10 mM benzamidine and membrane fractions sedimented by centrifugation at 15,000 g for 30 minutes at 4° C. Supernatants were removed and pellets extracted in 2% Triton X-114 extraction buffer for 2 hours on ice. Extracts were centrifuged at 15,000 g for 30 minutes at 4° C. and supernatants collected. The protein concentrations of extracts were determined using the Pearce BCA protein assay kit (Thermo Fisher Scientific).

For the competition assay, wheat germ agglutinin agarose eluates from extracts of ³⁵S-methionine-labelled GT1.7 cells were prepared as in section (3) above and diluted to 16×10⁶ cpm per ml. Aliquots (20 μl) of labelled GT1.7 cell glycoproteins were incubated with 5 μl of serum from patient 029 with or without 10 μl of detergent extracts of each mouse tissue equivalent to 100 μg of extracted protein for 18 hours at 4° C. Immune complexes were captured on protein A Sepharose and processed for SDS-PAGE and autoradiography as in (1) above.

The results are shown in FIG. 6. Reduced recovery of radiolabelled 38 kDa protein immunoprecipitated by the Glima 38 antibody positive 029 serum was observed in the presence of extracts of brain, pituitary and lung, indicative of competition by Glima 38 immunoreactivity in these tissue extracts (FIG. 6). These show that mouse brain, pituitary and lung are therefore potential sources of Glima 38 for purification and protein identification.

### EXAMPLE 5

Glima 38 Identification

The following strategy, based on the established biochemical properties of the protein and described in K. McLaughlin et al., 2016, Diabetes, Vol 65; 1-9, was used to identify Glima 38:
Sera from Type 1 diabetic patients were screened for Glima 38 antibodies using radiolabelled hypothalamic GT1.7 cells as source of antigen. High Glima 38 antibody-expressing serum samples were selected as a source of antibody for immunoaffinity purification (Example 3 above).
Extracts of mouse brain and lung as source of Glima 38 for purification, sequencing and protein identification (Example 4 above).
Mouse brain and lung samples were homogenised and membrane proteins extracted in Triton X-114 detergent
A hydrophobic membrane protein preparation was prepared by detergent phase separation at 30° C. The detergent pellet enriched in amphiphilic membrane proteins, including Glima 38, was collected for further purification.
Membrane glycoproteins were further enriched from the detergent phase pellet on wheat germ agglutinin beads.
Glima 38 was immunoprecipitated from the brain membrane glycoprotein extracts using a pool of Glima 38 antibody-positive patient sera. The brain glycoprotein preparation was also incubated with sera lacking Glima 38 antibodies as a negative control
The following samples were subjected to SDS poly acrylamide gel electrophoresis to separate proteins according to molecular weight:
   1 Brain glycoprotein preparation
   2 Lung glycoprotein preparation
   3 Glima 38 antibody positive sample immunoprecipitated proteins
   4 Glima antibody negative sample immunoprecipitated proteins
Gels were stained with Coomassie Brilliant Blue to visualise proteins on the gel. Regions of gels equivalent to a molecular 38 kDa bands were excised.
Proteins in gel slices were digested by incubation with trypsin.
The tryptic peptides were eluted and analysed by LC-MS/MS
The proteins common to samples 1, 2 and 3, but missing from sample 4 were considered candidates for Glima 38.
Candidates were tested by immunohistochemistry for appropriate tissue-specific expression
Candidates were tested for binding to autoantibodies in Type 1 diabetic patients' sera shown to be positive for Glima 38 antibodies in the GT1.7 cell assay.

A. Preparation of Protein Membrane Extracts

Mouse brain and lung were used as a source of Glima 38. One whole mouse brain or one pair of lungs were suspended in 10 ml ice-cold homogenisation buffer (10 mM Hepes, 250 mM sucrose, 10 mM benzamidine, pH 7.4) followed by homogenisation with 10 strokes in a Dounce homogeniser. Non-homogenised tissue and nuclear material was removed by centrifugation at 500×g for 5 minutes at 4° C. The supernatant was transferred to a fresh tube and centrifuged at 10,000×g for 15 minutes at 4° C. The supernatant containing the cytosolic fraction was removed and the pellet washed with 10 ml Hepes 5/5 (10 mM Hepes, 150 mM NaCl, 10 mM benzamidine, pH7.4) by centrifugation at 10,000×g for 15 minutes at 4° C. The pellets were then resuspended in 2% Triton X-114 diluted in Hepes 5/5 and incubated with agitation for 2 hours at 4° C. The suspension was centrifuged again at 10,000×g for 15 minutes at 4° C. and the supernatant containing the detergent extract was transferred to fresh tubes.

The detergent extract was phase separated by incubation at 30° C. for 10 min, followed by centrifugation at 1,000×g for 5 minutes at room temperature. The upper aqueous phase was removed and the detergent phase washed once in ice-cold Hepes 5/5 and re-extracted as before. Fractionation of the cytosolic and hydrophobic membrane extracts is shown in FIG. 7. Protein content of the final detergent phase was measured using the BCA assay (Pierce) and wheat germ-agarose was added at a ratio of 100 μl agarose by 5 mg total protein. The suspension was incubated overnight at 4° C. with gentle mixing. The supernatant was removed by centrifugation at 1,000×g for 1 minutes at 4° C. and the beads were washed twice with 0.5% Triton X-100 diluted in Hepes 5/5 and twice in NOG buffer (1% n-octyl-glucopyroside in Hepes 5/5). Wheat germ-binding proteins were eluted from the beads with 3×150 μl 0.5 M n-acetyl-glucosamine in NOG buffer. The samples were concentrated to 45 μl using the SDS-PAGE Sample Preparation kit (Pierce) and solubilised in SDS-PAGE sample buffer for 10 minutes at 60° C. Electrophoresis of samples (15 μl) was performed on a 12% Bis-Tris gel in MOPS running buffer for 72 minutes at 150 V with the Novex Sharp Pre-stained Marker used as a standard. Gels were fixed in 40% methanol/7% acetic acid for 30 minutes and stained with Brilliant Blue G-colloidal Coomassie for 2 h. Destaining was performed with 25% methanol/7% acetic acid for 5 min, followed by 25% methanol/2% acetic acid for the minimum period required to give good contrast against the background. The gel was stored in deionised water before gel slices corresponding to the 38 kDa region were excised.

B. Immunoaffinity Purification of Glima 38

For immunoaffinity purification, 250 μl pooled sera from three patients with high titre antibodies to Glima 38, and 250 μl pooled sera from three antibody-negative patients were used. Serum samples were incubated with Protein A-Sepharose for 1 hour at room temperature with rolling and washed three times in 1 ml borate buffer (100 mM boric acid, pH 8.3). Antibodies were cross-linked to the Protein A-Sepharose with 20 mM dimethylpimelidate in borate buffer for 1 hour at room temperature. The supernatant was then removed and unreacted sites were blocked with 20 mM ethanolamine for 10 minutes at room temperature. The antibody-crosslinked beads were then washed three times in borate buffer and twice in 0.5% Triton X-100 in Hepes 5/5.

Detergent phase prepared as described above from mouse brain (10 ml) was added to the Glima 38-positive Glima 38-negative antibody beads and incubated overnight at 4° C. with mixing. The beads were then washed three times with 0.5% Triton X-100 in Hepes 5/5 prior to the addition of SDS to a final concentration of 2% and incubated at 90° C. for 10 min. The supernatant was concentrated to 20 μl using the SDS-PAGE Sample Preparation kit (Pierce) and electrophoresis and gel staining was performed as above.

C. In-Gel Trypsinisation

Gel slices were processed using the In-gel Tryptic Digestion kit (Pierce). Gel slices were minced into 1 mm³ pieces and submerged in 40 μl destaining buffer and incubated for 30 minutes at 37° C. with agitation. The supernatant was removed and this step repeated once. Reducing buffer (30 μl) was then added and the samples were incubated at 60° C. for 10 minutes and allowed to cool. The supernatant was removed and replaced with 30 μl alkylation buffer and incubated for 1 hour in the dark at room temperature. The gel pieces were then washed twice in destaining buffer for 15 minutes at 37° C. with agitation.

Acetonitrile (50 μl) was added for 15 minutes at room temperature to shrink the gel pieces, and removed before briefly drying in the Speedivac. Ten microlitres of trypsin (10 ng/μl) was added to the gel pieces and incubated for 15 minutes at room temperature and then a further 25 μl of trypsin digestion buffer was added to cover the gel pieces and incubated at 30° C. overnight. The supernatant was collected into fresh tubes, and the gel pieces were re-extracted with 20 μl 2.5% trifluoroacetic acid for 15 minutes at room temperature, and the supernatant was combined with the first extract. The extracts were dried in the Speedivac and stored at −20° C. prior to mass spectrometric analysis.

D. LC-MS/MS and Data Analysis

Samples were reconstituted in 30 μl of 50 mM ammonium bicarbonate dissolved for 30 minutes at room temperature with constant agitation. Samples were centrifuged at 15,000×g for 15 minutes and the insoluble material was removed. Samples were transferred to autosampler tubes and 10 μl of each sample was analysed by LC-MS/MS.

Chromatographic separations were performed using an EASY NanoLC system (ThermoFisherScientific, UK). Peptides were resolved by reversed phase chromatography on a 75 μm C18 EASY column using a three step linear gradient of acetonitrile in 0.1% formic acid. The gradient was delivered to elute the peptides at a flow rate of 300 nL/min over 50 minutes per sample. The eluate was ionised by electrospray ionisation using an Orbitrap Velos Pro (ThermoFisherScientific, UK) operating under Xcalibur v2.2. The instrument was run in automated data-dependent switching mode, selecting precursor ions based on their intensity for sequencing by collision-induced fragmentation using a Top20 CID method. The MS/MS analyses were conducted using collision energy profiles that were chosen based on the mass-to-charge ratio (m/z) and the charge state of the peptide. Tandem mass spectra were processed into peak lists using Proteome Discoverer v1.3. Charge state deconvolution and deisotoping were not performed. All MS/MS samples were analyzed using Mascot (Matrix Science, London, UK; version 2.2.06). Mascot was set up to search the uniprot_sprot_130220 database (selected for Mus musculus, unknown version, 16597 entries) assuming the digestion enzyme trypsin. Mascot was searched with a fragment ion mass tolerance of 0.80 Da and a parent ion tolerance of 10.0 PPM. Oxidation of methionine and carbamidomethyl of cysteine were specified in Mascot as variable modifications. Each dataset was analysed with a reverse FASTA database acting as a decoy database.

Scaffold (version Scaffold_4.3.2, Proteome Software Inc., Portland, Oreg.) was used to validate MS/MS based peptide and protein identifications. Peptide identifications were accepted if they could be established at greater than 95.0% probability by the Peptide Prophet algorithm (Keller, A et al Anal. Chem. 2002; 74(20):5383-92). Protein identifications were accepted if they could be established at greater than 95.0% probability and contained at least 1 identified peptide. Protein probabilities were assigned by the Protein Prophet algorithm (Nesvizhskii, Al et al Anal. Chem. 2003; 75(17):4646-58). Proteins that contained similar peptides and could not be differentiated based on MS/MS analysis alone were grouped to satisfy the principles of parsimony.

Candidate proteins were searched on the UniProt website for characteristics including molecular weight, tissue distribution and glycosylation. Protein hits common to the brain and lung samples, and present in the antibody-positive immunoprecipitate, but not the antibody-negative immunoprecipitate, were further determined.

Fifty eight different proteins were detected in the brain 38 kDa band, 22 in the lung 38 kDa sample and 16 in Glima 38 antibody purification. Only 3 proteins were present in all samples except the negative control immunoprecipitate. These were:
Cytoplasmic actin-1—ubiquitous cytoskeletal protein. Not glycosylated. 42 kDa core protein
G(i) subunit alpha-2—widely expressed membrane-associated protein. Not glycosylated. 40 kDa core protein
Tetraspanin-7—Hydrophobic 4-transmembrane domain membrane glycoprotein expressed in neuroendocrine tissues. 5 glycosylation sites. 27 kDa core protein

Only Tetraspanin-7 has similar physical properties to Glima 38 and considered a candidate for Glima 38 identity and was therefore further investigated.

### EXAMPLE 6

Localisation of Tetraspanin-7 in Rodent and Human Tissues by Immunohistochemistry

Localisation of expression of Tetraspanin-7 in specific rodent and human tissues was performed using immunohistochemistry. Formalin fixed paraffin embedded rat brain, pituitary, adrenal gland, lung, thymus and pancreas were cut into 5 μM sections onto Superfrost plus slides (VWR). Sections were blotted and dried overnight. Prior to staining, sections were heated to 65° C. for 10 minutes and cooled. Sections were dewaxed by immersing for 10 minutes each in two changes of xylene. Xylene was removed and sections rehydrated by sequential 30 second washes in 100% ethanol, 95% ethanol and 70% ethanol. Sections were washed for 10 minutes in warm tap water. To break down cross links in the protein, the sections were placed in a ‘tender cook’ microwave pressure cooker, in pre-warmed citrate buffer (10 mM Citric Acid, 0.05% Tween 20, pH 6.0). The steamer was microwaved on full power until a constant stream of steam is visible, then steamed at full pressure for 3 min. The steamer was removed from the microwave oven, cooled for 10 min, and sections were removed and rinsed in water. A wax ring was drawn around each section to prevent cross contamination of antibodies, and endogenous peroxidases blocked, using 0.3% H2O2 diluted in PBS (137 mM NaCl, 4.3 mM Na2HPO4, 1.47 mM NaH2PO4 pH7) incubated for 5 minutes at room temperature. Sections were rinsed once in water, then non-specific binding blocked using swine serum (25% non-immune swine serum diluted in PBS), for 10 minutes at room temperature. Serum was tapped off onto a tissue and sections placed in a humidifying chamber for incubation with primary antibody.

The primary antibody (Anti TM4SF2, Sigma-Aldrich; HPA003140)) was applied at a concentration of 1/1,000 (diluted in PBS), and sections incubated overnight at 4° C. Sections were then washed three times in PBS, for 3 minutes per wash. A drop (50 μl) of secondary biotinylated link rabbit/mouse antibody (DAKO Envision kit) was applied for 15 minutes at room temperature. Sections were then washed three times in PBS, for 3 minutes per wash. Fifty microlitres of Streptavidin-HRP conjugate (DAKO Envision kit) was added for 15 minutes at room temperature. Sections were then washed three times in PBS, for 3 minutes per wash. DAB chromogen (DAKO Envision) was added at a concentration of 1/18 in diluent. Staining was visualized under a 10× magnification. Colour development was stopped by washing sections in water. Sections were then washed three times in PBS, for 3 minutes per wash. Sections were counterstained in Meyers Haematoxylin (Sigma-Aldrich; MHS1) for 2 min, then rinsed in water. Sections were dehydrated by passing through 5 minutes each in increasing concentrations of ETOH (70%, 95% and 100%) air dried for 5 min, then soaked for 5 minutes in xylene, prior to mounting using DPX (Sigma-Aldrich; 44581). Labelled sections were viewed under the microscope. Strong labelling was detected in the pancreatic islets of Langerhans, in the brain, pituitary and in regions of the lung (FIG. 8). The tissue distribution observed in these experiments was similar to that seen for Glima 38 in antibody blocking studies.

### EXAMPLE 7

Immunoprecipitation of Tetraspanin-7 from Mouse Brain with Human Sera

Three Glima 38 antibody-positive human sera and two Glima 38 antibody-negative sera (10 μl each) were each cross-linked to Protein A-Sepharose (10 μl) as described above. The antibody-bead complexes were then incubated overnight at 4° C. with 1.5 ml detergent extract from mouse brain, prepared as described above but using 2% Triton X-100 instead of Triton X-114. The pellets were washed three times in 0.5% Triton X-100 in Hepes 5/5 and solubilised in 10 μl 2×SDS sample buffer for 10 minutes at 60° C. Electrophoresis of samples (15 μl) was performed on a 12% polyacrylamide gel in MOPS running buffer for 72 minutes at 150 V with the Novex Sharp Pre-stained Marker used as a standard and transferred to a PVDF membrane for 1 hour at 30 V. The membrane was blocked in 5% milk in PBS/0.05% Tween-20 (PBS-T) for 1 hour at room temperature and then probed with anti-TM4SF2 (TSPAN7) produced in rabbit (Sigma-Aldrich; HPA003140) diluted 1/250 in 5% milk in PBS-T overnight at 4° C. The membrane was washed three times with 5% milk in PBS-T and probed with goat anti-rabbit IgG-peroxidase (Sigma-Aldrich; A0545). The membrane was washed a further three times with 5% milk in PBS-T and twice in PBS-T. SuperSignal West Pico Chemiluminescent Substrate was added to the membrane before exposure to X ray film and film development.

Tetraspanin-7 from mouse brain extracts was specifically immunoprecipitated by Glima 38 antibody-positive sera (FIG. 9), confirming that Tetraspanin-7 is the basic protein recognised by Glima 38 antibodies.

### EXAMPLE 8

Cloning of Mouse Tetraspanin-7

Full length mouse was PCR-amplified from cDNA of Min6 cells (a mouse islet cell line) using primers TSPAN7F (5′-ATGGCATCGAGGAGAATGG-3′) and TSPAN7R (5′-TTACACCATCTCATACTGATTGGC-3′). GoTaq polymerase (Promega) was used with a touchdown cycle: 95° C. for 2 minutes followed by 25 cycles of 95° C. for 30 sec, 60° C.-53° C. for 30 sec (−1° C. per cycle), 72° C. for 1 min, with a final extension step at 72° C. for 5 min. The PCR product was excised from a 1% agarose gel, purified using Freeze 'n Squeeze DNA Gel Extraction Spin Columns (BioRad) and ligated into the pGemT-Easy vector (Promega). The ligation reaction was transformed into XL-1 Blue E. coli cells. Plasmid DNA was extracted using the Quantum Prep Miniprep Kit (BioRad) and sequenced (Source BioScience).

In Vitro Transcription/Translation and Radioimmunoassay

Tetraspanin-7 pDNA was transcribed and translated in vitro in the presence of ³⁵S-methionine using the TNT®-Quick-Coupled Transcription/Translation System (Promega, Southampton, UK). Incorporated radioactivity was determined by precipitation of the translated protein with 10% trichloroacetic acid, followed by scintillation counting. Radiolabelled protein (20,000 cpm in 20 μl) was incubated with 5 μl of test sera for 16 hours at 4° C. in wash buffer (10 mM HEPES, pH7.4, 150 mM NaCl, 20 mM methionine, 0.5 mg/ml BSA and 0.5% Triton X-100). Immune complexes were immunoprecipitated with Protein A-Sepharose and washed five times under vacuum filtration with wash buffer, followed by two washes in water. The quantity of immunoprecipitated radiolabelled antigen was determined by liquid scintillation counting. Specific binding of radiolabelled-Tetraspanin-7 by Glima 38 antibody-positive sera was not observed, possibly due to misfolding of the in vitro transcribed and translated protein (FIG. 10). Additional radioimmunoassay experiments were carried out using canine pancreatic microsomes in the transcription/translation reactions (0.5-1.5 μl/reaction) to try to overcome this problem, but clear evidence of binding was still not detected (FIG. 11).

Cloning of Mouse Tetraspanin-7 into pFLAG Vector for Expression in E. coli

Because of the difficulties in expressing immunoreactive Tetraspanin-7 in vitro, the sequence was cloned into a vector for expression in E. coli.

To insert into the expression vector (pFLAG-CTS expression vector; Sigma-Aldrich), Tetraspanin-7 was re-amplified from the TSPAN7-pGEMT-Easy contruct using primers TSPAN7EcoRIF (5′-GAATTCATGGCATCGAGGAGAATGG-3′) and TSPAN7BglIIR (5′-AGATCTCACCATCTCATACTGATTGGC-3′) to introduce an EcoR1 site at the 5′ end and a Bg1II site at the 3′ end with the native stop codon removed to allow expression of the C-terminal FLAG tag from the expression vector. PCR conditions were as previously described, but a constant annealing temperature of 55° C. was used. The PCR product was excised, purified and ligated into pGemT-Easy as before. One microgram of plasmid DNA and 1 μg of pFLAG-CTS were digested with EcoRI and Bg1II in Buffer D (Promega) for 3 hours at 37° C. The products were gel-purified and the Tetraspanin-7 insert was ligated overnight into the expression vector at a 3:1 ratio using T4 DNA Ligase (Promega). The ligation reaction was transformed into XL-1 Blue E. coli cells. Plasmid DNA was extracted and re-transformed into BL21 E. coli cells for expression.

For protein expression, a 5 ml overnight culture of transformed BL21 cells was inoculated into 500 ml culture of LB broth containing ampicillin and incubated at 37° C. with shaking. When the OD600=1, expression was induced with 1 mM IPTG and the culture was further incubated at 25° C. overnight. The cells were centrifuged at 10,000×g for 10 minutes at 4° C. and stored at −20° C. before extraction.

Protein was extracted from the frozen pellet in 15 ml extraction buffer (1×PBS, 10 mM benzamidine, 1 mM PMSF) with Hen Egg Lysozyme (1 mg/ml) for 30 minutes at room temperature with agitation. Triton X-100 was added to a final concentration of 0.1% for 5 minutes followed by 800 U of DNase for 10 min. The lysate was centrifuged at 10,000×g for 10 minutes at 4° C. and the supernatant stored at 4° C.

Immunoprecipitation of Recombinant Tetraspanin-7 with Human Sera

Ten microlitres of human sera was bound to 10 μl of packed Protein A-Sepharose for 1 hour at room temperature with agitation. The beads were washed three times with 0.5% Triton X-100 in Hepes 5/5. Lysate from Tetraspanin-7-transformed

E. coli (1.5 ml) was added to each antibody-bead complex and incubated overnight at 4° C. with agitation. The beads were washed three times with 0.5% Triton X-100 in Hepes 5/5 and resuspended in 10 μl 2×SDS sample buffer and heated for 10 minutes at 60° C. The samples (10 μl) were resolved on a 12% polyacrylamide gel run at 150 V for 72 minutes with MOPS running buffer and transferred to a PVDF membrane for 1 hour at 30 V. The membrane was blocked in 5% milk in PBS-T for 1 hour at room temperature and probed with anti-Tetraspanin-7 antibody as previously described.

Recombinant Tetraspanin-7 was also specifically immunoprecipitated by Glima 38 antibody-positive sera (FIG. 12), confirming our previous observation that Tetraspanin-7 is the basic protein recognised by Glima 38 antibodies.

### EXAMPLE 9

Analysis of Tetraspanin-7 Antibodies by Luminescence Immunoprecipitation Assay System (LIPS)

As described in K. McLaughlin et al., 2016, Diabetes, Vol 65; 1-9, patients screened for Glima 38 antibodies were analyzed for Tspan7 antibodies by immunoprecipitation of recombinant NanoLuc-tagged human Tetraspanin-7. Western blotting with rabbit polyclonal antibodies to both nanoluciferase and Tetraspanin-7 detected diffuse 38,000 Mr bands (the expected size of the nonglycosylated fusion protein) as the dominant immunoreactivity in cells transfected with the construct, with additional bands at approximately 80,000 Mr (FIG. 13A). The 38,000 Mr protein partitioned into the detergent on temperature-induced phase separation in Triton X-114. Transfected cell extracts were used in immunoprecipitation studies with normal control sera or with sera from Glima antibody-positive and Glima antibody-negative patients with type 1 diabetes. All but one of the controls (control sample V015) (n=52) had low levels of Tetraspanin-7 antibodies (FIG. 13B). Four patients with high levels of Glima antibodies (FIG. 5) also immunoprecipitated high luciferase activity in the Tetraspanin-7 antibody assay (FIG. 13B), and significantly higher levels of Tetraspanin-7 antibodies were found in Glima antibody-positive patients than Glima antibody-negative patients (P, 0.0001; Mann-Whitney U test). In competition assays, natural or recombinant Tetraspanin-7 in brain or E. coli extracts partially (control sample V015) or completely (Glima antibody-positive patients with type 1 diabetes) blocked antibody binding to the NanoLuc-Tetraspanin-7 construct (FIG. 13C). Control sample V015 did not bind Tetraspanin-7 from mouse brain extracts when tested in the Western blotting assay. A second set of 94 patients with recent onset of type 1 diabetes was also tested in the Tetraspanin-7 antibody assay. Using a mean cutoff 63 SDs of controls (omitting the outlier), 40 (43%) were positive for Tetraspanin-7 antibodies (FIG. 13B). This LIPS confirms that patients with high levels of Glima autoantibodies were also strongly positive for anti-Tetraspanin-7 antibodies.

### EXAMPLE 10

Historical serum samples, obtained from an individual at risk of developing diabetes and which were stored after sample collection at −20° C., were examined for antibodies to Tetraspanin-7 using the LIPS methodology as described in Example 9. The individual developed diabetes in February 1992.

The samples were also tested for the presence of antibodies to another islet autoantigen, IA-2.

The results are shown in FIG. 14. Antibodies to tetraspanin-7 were detected more than two years before diabetes onset and at least one year before appearance of antibodies to the other islet autoantigen, IA-2. This indicates that the detection of anti-Tetraspanin-7 antibodies has predictive value in determining a predisposition to Type 1 diabetes, and may have advantages over IA-2 as a diabetes marker.

1. A method for the diagnosis of Type 1 diabetes, or a predisposition towards Type 1 diabetes, or to monitor the efficacy of a therapy to prevent or treat Type 1 diabetes, said method comprising contacting a sample from a subject with a reagent selected from Tetraspanin-7 or a fragment, or a modified form thereof, and detecting an interaction indicative of the presence of an autoimmune response to Tetraspanin-7.

2. The method of claim 1 wherein the autoimmune response detected is the presence of autoantibodies to Tetraspanin-7.

3. The method of claim 2 wherein the level of autoantibodies present are measured and compared to a normal reference range.

4. The method of claim 2 wherein the reagent comprises Tetraspanin-7 or a fragment thereof, modified to increase the efficiency of detection of antibody binding.

5. The method according to claim 2 wherein the reagent is complexes to a further protein or peptide which increases the efficiency of antibody detection.

6. The method according to claim 2 wherein the autoantibodies are detected using an assay selected from radioimmunoprecipitation assay, ELISA, time-resolved fluorescence assay, and a luminescence assay, including a luminescence immunoprecipitation assay.

7. The method of claim 1 wherein the autoimmune response detected is the presence of Tetraspanin-7 specific T-cells.

8. The method of claim 7 wherein Tetraspanin-7 specific T-cells are detected by means of an assay selected from a T cell proliferation assay, a binding assay using soluble MHC tetramers, a binding assay using soluble T cell receptors, an ELISPOT assay or an assay based upon cytokine detection.

9. The method of claim 8 wherein the assay is a MHC-tetramer binding assay.

10. The method of claim 1 which further comprises the steps detecting an immune response to one or more of GAD, insulin, IA-2 or ZnT8.

11. The method of claim 10 wherein the further steps comprise detecting antibodies to one or more of GAD, insulin, IA-2 or ZnT8.

12. A kit for use in the method of claim 1, said kit comprising Tetraspanin-7 or a fragment, or a modified form thereof, and means for detecting an interaction indicative of the presence of an autoimmune response to Tetraspanin-7.

13. A method for detecting an autoimmune response to Tetraspanin-7, said method comprising contacting Tetraspanin-7 or fragments, in particular epitopic fragments, thereof or modified forms thereof with a sample obtained from a subject having or suspected of having Type 1 diabetes or a predisposition towards Type 1 diabetes.

14. The method of claim 13 which further comprises administering Tetraspanin-7 or an epitopic fragment thereof or modified form thereof to the subject for prophylactic or therapeutic treatment of Type 1 diabetes.

15. A fragment or modified form of Tetraspanin-7.

16. A pharmaceutical composition comprising Tetraspanin-7 or fragments thereof, or modified forms of these in combination with a pharmaceutically acceptable carrier or excipient.

17. A method for preventing Type 1 diabetes, delaying the onset of Type 1 diabetes, or ameliorating autoimmunity in an individual with Type 1 diabetes (including ameliorating any one or more symptoms of the disease), comprising administering to an individual in need thereof, an agent that either (i) elicits a Tetraspanin-7-specific immune response that protects [beta] cells of the pancreatic islet in the patient; or (ii) targets Tetraspanin-7-specific T cells in the individual, and induces necrosis or apoptosis of the Tetraspanin-7-specific T cells; or (iii) induces tolerance of Tetraspanin-7 T cells in the individual; or (iv) depletes Tetraspanin-7 specific B-cells.
